(19) United States
(12) Patent Application Publication
(10) Pub. No.: US 2006/0252804 A1
(43) Pub. Date: Nov. 9, 2006

(54) Flupirtin injectable galenic form

(75) Inventors: Michael Pieroth, Weinbohla (DE); Norbert Stang, Oberhermsdorf (DE); Rudy Thoma, Luhnsdorf (DE); Henning Blume, Bad Homburg (DE)

Correspondence Address: GUDRUN E. HUCKETT DRAUDT, LONSSTR. 53, WUPPERTAL, 42289, DE

(73) Assignee: AWD.pharma GMBH &Co. KG, Dresden (DE)

(21) Appl. No.: 10/560,420

(22) PCT Filed: Jun. 16, 2004
(86) PCT No.: PCT/EP04/06449

§ 371 Date: Jun. 8, 2006

(30) Foreign Application Priority Data
Jun. 20, 2003 (DE) 10327674.2

(51) Int. Cl. A61K 31/44

(52) U.S. Cl. 514/352

(57) ABSTRACT

A lyophilisate having as an active ingredient flupirtine in base form or as a physiologically tolerated salt is suitable for producing a pharmaceutical composition for parenteral administration. The physiologically tolerated salt is an acid addition salt of flupirtine. The lyophilisate contains at least one additive selected from the group consisting of a cake-forming agent, an antioxidant, and a detergent. In a process for producing a flupirtine-containing pharmaceutical composition for parenteral administration, a flupirtine-containing lyophilisate is dissolved in at least one solvent that is an aqueous medium or an organic solvent or a combination thereof to obtain a liquid pharmaceutical composition ready for use. In a process for producing a flupirtine-containing lyophilisate, a flupirtine solution is prepared by dissolving flupirtine base in an aqueous medium and the flupirtine solution is freeze dried.

The present invention relates to a flupirtine-containing lyophilisate, to the use of the lyophilisate for producing a pharmaceutical composition for parenteral administration, to a process for producing a flupirtine-containing pharmaceutical composition for parenteral administration, to a process for producing the flupirtine-containing lyophilisate, and to the flupirtine-containing pharmaceutical composition produced using the lyophilisate.

Flupirtine (Katadolon®; 2-amino-3-carbethoxyamino-6-(4-fluorobenzylamino)pyridine=2-amino-3-ethoxycarbonylamino-6-(p-fluorobenzylamino)pyridine) is a centrally acting, non-opiate analgesic. Flupirtine, especially in the form of its maleic acid salt, has been employed successfully for many years for the therapy of, for example, neuralgias, pain associated with degenerative joint diseases, headaches and postoperative pain. According to DE 41 22 166 A1, flupirtine can also be employed as agent for controlling disorders or pathological symptoms which are based on muscle tenseness or are a consequence of such muscle tenseness. DE 43 27 516 furthermore describes the use of flupirtine for treating cerebral ischemia and neurodegenerative disorders. DE 195 41 405 A1 discloses the use of flupirtine for the prophylaxis and therapy of disorders associated with impairment of the haematopoietic cell system. DE 100 48 969 A1 further describes the use of flupirtine for tinnitus treatment. The preparation of flupirtine and physiologically usable salts thereof is described in DE 17 95 858 C2, DE 31 33 519 C2 and

DE 34 16 609 A1.

Flupirtine is mainly administered orally. Thus, DE 93 21 574 U1 describes, for example, pharmaceutical formulations in the form of tablets, granules or pellets which comprise flupirtine maleate as active ingredient. DE 43 19 649 A1 discloses solid flupirtine-containing oral dosage forms with controlled delivery of active ingredient.

However, because of the good analgesic effect of flupirtine, it is desirable to administer flupirtine parenterally in order to achieve a rapid local or systemic effect. The obstacle to this is, however, that flupirtine and physiologically active salts thereof are scarcely soluble in aqueous solutions and in most physiologically tolerated organic solvents.

DE 34 16 609 A1 describes pharmaceutical formulations in the form of injectable flupirtine gluconate solutions which are prepared using suitable solvents. The solvent employed is in particular a mixture of polyethylene glycol and water or a mixture of glycofurol and water. However, the described injection solutions have a number of serious disadvantages. Thus, the flupirtine gluconate solutions prepared using the polyethylene glycol/water or glycofurol/water mixtures are extremely hypertonic and therefore suitable only for intramuscular use. Owing to the relatively low pH of 3.2 to 3.6 and the excipients employed, such as sodium disulphite and propylene glycol, irritation at the administration site is also common. In addition, the described flupirtine gluconate solutions have inadequate physical stability because they are stable only over a very limited period and, after only a few weeks, there is onset of a precipitation process which distinctly limits the shelf life of the finished product. In addition, it has emerged that the physical stability of the flupirtine solutions depends to a large extent also on the storage temperature. Since the onset of precipitation is distinctly earlier at lower temperatures, it is necessary to maintain a minimum temperature of 20° C. during storage of the flupirtine solutions in order to improve the storage stability. Ideally, ampoules containing such solutions for injection ought to be stored at temperatures of 25° C. to 30° C., but this is scarcely possible in practice.

The technical problem on which the present application is based is therefore to provide means and processes for producing flupirtine-containing pharmaceutical compositions suitable for parenteral administration and not having the disadvantages, known in the state of the art, of parenteral pharmaceutical compositions, that is to say in particular do not cause side effects such as irritation on administration and, in addition, are physically and chemically stable over a sufficiently long period.

The present invention solves the technical problem on which it is based through provision of a lyophilisate which comprises the active ingredient flupirtine in base form or as physiologically tolerated salt and which can be employed to produce a pharmaceutical composition for parenteral administration.

It has surprisingly been found according to the invention that the disadvantages and problems known in the state of the art to be associated with the handling and storage of flupirtine solutions for injection can be completely eliminated through the use of the flupirtine lyophilisate according to the invention for producing liquid formulations for parenteral administration. Thus, reconstitution of the flupirtine lyophilisate according to the invention advantageously leads to very clear flupirtine solutions which are stable for several hours and show no precipitation processes, whereas, with conventional solutions for injection, flupirtine precipitates immediately after the onset of dissolution. Owing to their stability, the flupirtine solutions produced using the flupirtine lyophilisates according to the invention can therefore be employed in an excellent manner for parenteral administration, especially as solutions for injection or infusion.

A further surprising advantage of the flupirtine lyophilisates according to the invention is that purely aqueous media can be employed to produce liquid dosage forms. Whereas only solvent systems which have a high content of organic solvents such as propylene glycol, but not pure aqueous media, can be used to produce conventional flupirtine solutions for injection, the flupirtine lyophilisates according to the invention are outstandingly soluble in aqueous systems, so that no organic solvents or solubilizing substances need to be employed for dissolving. The flupirtine lyophilisates according to the invention additionally have the advantage that heating is unnecessary in the dissolving of the lyophilisate, because the flupirtine lyophilisates according to the invention dissolve very rapidly even at room temperature.

A further advantage is that the lyophilisates according to the invention can be reconstituted and/or diluted as desired. Thus, the flupirtine lyophilisate according to the invention can be employed equally for producing solutions for intramuscular or intravenous injection, but also for preparing solutions for infusion. It is also possible in this connection for the reconstituted aqueous preparation to be used as admixture to solutions conventionally used for infusion. This form of use is advantageous in particular for those patients requiring systemic pain treatment in association with other therapeutic procedures. Since the lyophilisates according to the invention characteristically dissolve very rapidly, the lyophilisates according to the invention can be reconstituted immediately before use.

In connection with the present invention, a “lyophilisate” means a material which is obtained by drying in the deep-frozen state under high vacuum through freezing of the solvent, which evaporates in the frozen state. A freeze-dried material obtained in this way is very porous and retains its original volume. Metabolic functions, enzyme functions and/or biological activity of the material cease after lyophilization.

In connection with the present invention, a “pharmaceutical composition” or a “medicament” means a mixture which is used for diagnostic, therapeutic and/or prophylactic purposes, that is to say one which promotes or restores the health of a human or animal body, and which includes at least one natural or synthetically prepared active ingredient which causes the therapeutic effect.

The pharmaceutical composition may include additives normally used in the specialist field, for example stabilizers, production aids, release agents, emulsifiers, detergents, antioxidants, cake-forming agents or other substances used for producing pharmaceutical compositions, in particular for producing liquid dosage forms.

In a preferred embodiment of the invention, the pharmaceutical composition to be produced according to the invention is a liquid pharmaceutical composition for parenteral administration.

A “dosage form or pharmaceutical composition for parenteral administration” means a sterile pharmaceutical composition which is administered with avoidance of the gastrointestinal tract. The advantages of parenteral administration, especially compared with oral administration, are in particular that a very rapid onset of action is possible, that side effects such as, for example, vomiting or gastrointestinal irritation are very substantially avoided, that active ingredients do not undergo gastrointestinal inactivation, that it is also possible to administer active ingredients which are generally inadequately absorbed from the gastrointestinal tract, that the blood level of the administered active ingredient can be calculated beforehand, and that the so-called first pass effect is avoided.

Pharmaceutical compositions for parenteral administration are, in particular, solutions for injection and infusion. “Injections” or “solutions for injection” are preparations with small volumes, in particular between 1 and 20 ml, which are administered as solution, suspension or emulsion. With an “infusion” or “solution for infusion”, volumes larger than 100 ml are administered. The commonest parenteral administration routes are intravenous (i.v.), intramuscular (i.m.) and subcutaneous (s.c.) administration. Intravenous administration makes it possible for active ingredients which have tissue-irritant effects with other parenteral administration routes to be supplied and administered rapidly. With intramuscular and subcutaneous injections it is necessary to take account of isohydria and isotonicity because, otherwise, local signs of intolerance may appear.

The invention therefore provides for the pharmaceutical composition for parenteral administration which is to be produced using the lyophilisate according to the invention to be a solution for injection or solution for infusion.

The invention provides for flupirtine to be present in the lyophilisates according to the invention possibly either as base or as physiologically tolerated salt, with, in a preferred embodiment of the invention, the lyophilisate according to the invention comprising at least 100 mg of flupirtine, this stated amount being based on flupirtine base.

“Physiologically tolerated salts” of flupirtine mean in particular those flupirtine salts which are in the form of acid addition salts and which have a therapeutic index which is characterized by a sufficiently large distance between the sensitivity curves of the flupirtine salts for their therapeutic and lethal effect.

Examples of suitable acids for preparing physiologically tolerated flupirtine salts include hydrohalic acids, sulphuric acid, phosphoric acids, nitric acid, perchloric acid, organic mono-, di- or tricarboxylic acids of the aliphatic, alicyclic, aromatic or heterocyclic series, and sulphonic acids. Preferred examples of suitable acids are formic, acetic, propionic, succinic, glycolic, lactic, malic, tartaric, citric, ascorbic, maleic, fumaric, hydroxymaleic, pyruvic, phenylacetic, benzoic, p-aminosalicylic, embonic, methanesulphonic, ethanesulphonic, hydroxyethanesulphonic, ethylenesulphonic, halobenzenesulphonic, toluenesulphonic, naphthalenesulphonic acids, sulphanilic acid and hydrochloric acid. Particularly preferably employed according to the invention for preparing the physiologically tolerated flupirtine salt is gluconic acid.

In a preferred embodiment of the invention, the physiologically tolerated flupirtine salt is therefore the formate, acetate, propionate, succinate, glycolate, lactate, malate, tartrate, citrate, maleate, fumarate, pyruvate, phenylacetate, benzoate, embonate, methanesulphonate, ethanesulphonate, hydroxyethanesulphonate, ethylenesulphonate, halobenzonesulphonate, toluenesulphonate, naphthalenesulphonate, aminobenzenesulphonate or chloride of flupirtine. In a particularly preferred embodiment of the invention, the physiologically tolerated flupirtine salt is flupirtine gluconate.

The invention further provides for the flupirtine lyophilisate to comprise the acidic constituent of the physiologically tolerated flupirtine salt in an amount of from 60 mg to 650 mg, preferably from 200 mg to 400 mg, based on 100 mg of flupirtine.

In a preferred embodiment of the invention, the flupirtine lyophilisate according to the invention additionally comprises at least one cake-forming agent. A “cake-forming agent” or “bulking agent” means an agent which assists the formation of a porous cake with a very large internal surface area during and/or after lyophilization of a material. In a preferred embodiment, the flupirtine lyophilisate according to the invention comprises mannitol, sucrose or glycine as cake-forming agent. The invention provides in particular for the content of cake-forming agent in the flupirtine lyophilisate according to the invention to be an amount of from 10 mg to 1000 mg, preferably from 30 mg to 300 mg, based on 100 mg of flupirtine.

A further preferred embodiment of the invention provides for the flupirtine lyophilisate according to the invention to comprise additionally at least one antioxidant. “Antioxidants” mean excipients able to inhibit, delay or suppress oxidation of a substance, in particular of an active ingredient. Antioxidants may be free-radical scavengers, easily oxidizable substances or synergists. Free-radical intermediates are often produced in the oxidation of organic compounds.

Excipients with sterically hindered phenolic groups can easily transfer hydrogen radicals to these intermediates, themselves forming more stable molecules. This interrupts the oxidative chain reaction. Free-radical scavengers are employed in particular in non-aqueous, lipophilic systems. By contrast, easily oxidizable substances are mainly employed in aqueous systems, making use of the fact that every substance to be protected has a particular electrical oxidation potential. The antioxidant to be employed in this case has a distinctly lower oxidation potential than the substance to be protected. In the presence of oxygen, the antioxidant is then more easily oxidized than the substance to be protected. At the same time, the easily oxidizable excipient acts as proton donor and thus stabilizing. Pharmaceutically utilizable substances of this type are, in particular, ascorbic acid with a standard oxidation potential of −0.04 V. Bisulphites and sulphites have a standard oxidation potential of +0.12 V. Synergists comprise a group of excipients which assist the action of antioxidants either by regeneration of excipient molecules which have already been oxidized, by complexation of traces of heavy metals, by decomposition of peroxides as intermediates of the oxidation or by setting up an oxidation-inhibiting pH.

In a preferred embodiment of the invention, the lyophilisate according to the invention comprises sodium bisulphite or ascorbic acid as antioxidant. The invention provides for the antioxidant to be present in the flupirtine lyophilisate according to the invention in an amount of from 0.5 mg to 10 mg, particularly preferably in an amount of from 2 mg to 5 mg, based on 100 mg of flupirtine.

A further preferred embodiment of the invention provides for the flupirtine lyophilisate according to the invention additionally to comprise at least one detergent. In connection with the present invention, a “detergent” means an organic surface-active substance which may have an anionic, cationic, ampholytic or nonionic structure. Detergents are also referred to as surfactants. In pharmacy, cationic surfactants are mainly employed as preservatives or disinfectants. Surfactants may also be employed as W/O or O/W emulsifiers, wetting agents, solubilizers, foam stabilizers or antifoams. The selection of detergents for particular tasks depends both on the chemical constitution of the hydrophilic and lipophilic groups of the compound employed as detergent, because they determine the affinities for the phases which are present, and on the HLB values, but also on the melting or solidification points and on the viscosities.

In a preferred embodiment, the lyophilisate according to the invention comprises a polyvinylpyrrolidone as detergent. Polyvinylpyrrolidones are products of the polymerization of vinylpyrrolidone. A series of fractions with different molecular sizes or molecular chain lengths is commercially available. A prominent property of polyvinylpyrrolidones is the good solubility both in water and in polar organic solvents such as alcohols, glycols, etc. The invention provides in particular for the detergent, in particular polyvinylpyrrolidone, to be present in the flupirtine lyophilisate according to the invention in an amount of from 10 mg to 150 mg, particularly preferably in an amount of from 10 mg to 50 mg, based on 100 mg of flupirtine.

The present invention likewise relates to the use of the flupirtine-containing lyophilisate according to the invention for producing a pharmaceutical composition for parenteral administration. The invention provides in this connection for the lyophilisate to be used for producing the pharmaceutical composition for parenteral administration by dissolving the lyophilisate in an aqueous medium and/or an organic solvent, resulting in the pharmaceutical composition for parenteral administration. The invention particularly provides for the lyophilisate to be dissolved at room temperature for this purpose. The aqueous medium preferably used according to the invention is water, particularly preferably water for injections. Another embodiment of the invention provides for using a suitable buffer solution as aqueous medium. A further embodiment provides for the lyophilisate to be dissolved in a water/solvent mixture to produce the parenteral pharmaceutical composition.

The present invention likewise relates to a process for producing a flupirtine-containing pharmaceutical composition for parenteral administration, where a flupirtine-containing lyophilisate according to the invention is dissolved in an aqueous medium and/or an organic solvent, and a liquid pharmaceutical composition ready for use is obtained.

The flupirtine lyophilisate according to the invention is preferably dissolved at room temperature. In a particularly preferred embodiment, the flupirtine lyophilisate according to the invention is dissolved in water, in particular water for injections. The flupirtine lyophilisate can, however, also be dissolved in a buffer solution or in a water/solvent mixture.

The isotonicity of the resulting solution can be adjusted via the volume of the aqueous medium used for dissolving. Before administration of the parenteral pharmaceutical composition to be prepared, a decision can be made as to how the lyophilisate is to be reconstituted and whether dilution can take place where appropriate. Thus, it is equally possible to prepare an intramuscular or intravenous injection from the lyophilisate according to the invention. The reconstituted aqueous preparation can also be used as admixture to conventional solutions for infusion.

The invention therefore provides for the pharmaceutical composition for parenteral administration which is produced using the process according to the invention to be a solution for injection. If the solution which is to be produced for injection is to be administered intravenously, the invention provides for the lyophilisate according to the invention, which preferably contains 100 mg of flupirtine, to be dissolved in from 3 to 20 ml, preferably 9 to 15 ml, of water for injections, buffer solution or water/solvent mixture. If the solution which is to be produced for injection is to be administered intramuscularly, the invention provides for the lyophilisate according to the invention, which preferably contains 100 mg of flupirtine, to be dissolved in 3 ml of water for injections, buffer solution or water/solvent mixture. A further preferred embodiment of the invention provides for the pharmaceutical composition for parenteral administration to be a solution for infusion.

The present invention likewise relates to a process for producing the flupirtine-containing lyophilisate according to the invention, comprising
a) preparation of a flupirtine solution by adding flupirtine base to an aqueous medium and dissolving therein, and
b) freeze drying of the resulting flupirtine solution.

The invention thus provides for flupirtine initially to be dissolved in base form in the first step in an aqueous medium. If the flupirtine lyophilisate to be prepared is to comprise exclusively flupirtine base, the flupirtine base is preferably dissolved in water, in particular water for injections. If the flupirtine lyophilisate to be prepared is to comprise a physiologically tolerated flupirtine salt, the flupirtine base is dissolved in an aqueous solution of the appropriate acid, the acid being selected from the group consisting of gluconic, formic, acetic, propionic, succinic, glycolic, lactic, malic, tartaric, citric, ascorbic, maleic, fumaric, hydroxymaleic, pyruvic, phenylacetic, benzoic, p-aminosalicylic, embonic, methanesulphonic, ethanesulphonic, hydroxyethanesulphonic, ethylenesulphonic, halobenzenesulphonic, toluenesulphonic, naphthalenesulphonic acids, sulphanilic acid and hydrochloric acid. In a preferred embodiment, the lyophilisate to be prepared is to contain flupirtine gluconate. A lyophilisate comprising flupirtine gluconate is therefore prepared by dissolving the flupirtine base in a gluconic acid solution.

In a preferred embodiment of the invention, the aqueous medium, for example water or the acid solution, used to dissolve the flupirtine base is heated to a temperature above room temperature, and kept at this temperature, before addition of the flupirtine base. Only after the heating is flupirtine added to the heated aqueous medium and dissolved therein. In a preferred embodiment, the aqueous medium is heated to a temperature of from 30° C. to 90° C., particularly preferably 70° C. The invention further provides for flupirtine to be added with stirring to the preferably heated aqueous medium. The solution is then stirred until flupirtine has completely dissolved.

The invention further provides for the flupirtine solution prepared in this way subsequently to be filtered. A filter with a pore width of 0.2 □m is particularly preferably employed in this connection. The preferably filtered flupirtine-containing solution is then introduced into freeze-drying bottles, which are then provided with freeze-drying stoppers. The flupirtine solution is frozen by storing the freeze-drying bottles at −45° C.

The invention provides for the actual freeze drying to comprise a main drying and an after-drying. In a preferred embodiment, the main drying takes place at a temperature of from −37° C. to −23° C. under a pressure of from 10 to 100 mbar. In a preferred embodiment, the subsequent after-drying takes place at a temperature of 27° C. under a pressure of 0.0001 mbar. After the freeze drying, the freeze dryer is destressed with N₂. Sterile closure of the bottles containing the flupirtine lyophilisate then preferably takes place under a nitrogen atmosphere.

The present invention likewise relates to the liquid pharmaceutical composition for parenteral administration which is obtainable through the flupirtine lyophilisate according to the invention.

The present invention is explained in more detail by the following examples.

### EXAMPLE 1

### Production of a Flupirtine-Containing Lyophilisate

7.81 g of gluconic acid d-lactone are dissolved in 70 ml of water and then heated to 70° C. while stirring at this temperature, 3.33 g of flupirtine are added and stirred until completely dissolved. The mixture obtained in this way is filtered through a filter with a pore width of 0.2 □m. After the filtration, the solution is dispensed into freeze-dried bottles and provided with suitable freeze-drying stoppers. The contents are frozen by storing the bottles at −45° C. The main drying of the freeze-drying process takes place at −37° C. to −23° C. under 100 to 10 mbar. The after-drying is then carried out at 27° C. under 0.0001 mbar. After the drying, the freeze dryer is destressed with N₂. The bottles are then closed under a nitrogen atmosphere.

### EXAMPLE 2

### Production of a Flupirtine-Containing Lyophilisate

7.81 g of gluconic acid d-lactone are dissolved in 70 ml of water and then heated to 70° C. while stirring at this temperature, 4.0 g of mannitol and 3.33 g of flupirtine are added and stirred until the added compounds have completely dissolved. The mixture obtained in this way is filtered through a filter with a pore width of 0.2 □m. After the filtration, the solution is dispensed into freeze-drying bottles and provided with suitable freeze-drying stoppers. The contents are frozen by storing the bottles at −45° C. The main drying of the freeze-drying process takes place at −37° C. to −23° C. under 100 to 10 mbar. The after-drying is then carried out at 27° C. under 0.0001 mbar. After the drying, the freeze dryer is destressed with N₂. The bottles are then closed under a nitrogen atmosphere.

### EXAMPLE 3

### Production of a Flupirtine-Containing Lyophilisate

7.81 g of gluconic acid d-lactone are dissolved in 70 ml of water and then heated to 70° C. while stirring at this temperature, 7.5 g of sucrose, 0.4 g of polyvinylpyrrolidone (MW approximately 11 500; collidone PF17; BASF) and 3.33 g of flupirtine are added. The solution is stirred until the added substances have completely dissolved. The mixture obtained in this way is filtered through a filter with a pore width of 0.2 □m. After the filtration, the solution is dispensed into freeze-drying bottles and provided with suitable freeze-drying stoppers. The contents are frozen by storing the bottles at −45° C. The main drying of the freeze-drying process takes place at −37° C. to −23° C. under 100 to 10 mbar. The after-drying is then carried out at 27° C. under 0.0001 mbar. After the drying, the freeze dryer is destressed with N₂. The bottles are then closed under a nitrogen atmosphere.

### EXAMPLE 4

### Composition of a Flupirtine Lyophilisate

One Bottle Contains:

100.00 mg of flupirtine
257.85 mg of gluconic acid
300.00 mg of mannitol.

### EXAMPLE 5

### Composition of a Flupirtine Lyophilisate

One Bottle Contains:

100.00 mg of flupirtine
257.85 mg of gluconic acid
225.00 mg of sucrose
12.00 mg of polyvinylpyrrolidone
   (Kollidon 17 PF, BASF).

### EXAMPLE 6

### Composition of a Flupirtine Lyophilisate

One Bottle Contains:

100.00 mg of flupirtine
257.85 mg of gluconic acid
120.00 mg of mannitol
4.50 mg of sodium bisulphite

### EXAMPLE 7

### Composition of a Flupirtine Lyophilisate

One Bottle Contains:

100.00 mg of flupirtine
257.85 mg of gluconic acid
207.00 mg of mannitol

### EXAMPLE 8

### Production of a Liquid Pharmaceutical Composition

3 ml of water for injections is added to a bottle of the lyophilisate described in Example 7. After occasional swirling, a clear solution is obtained after about 1 min. The solution is distinctly hypertonic at 980 mosmol/kg.

### EXAMPLE 9

### Production of a Liquid Pharmaceutical Composition

9 ml of water for injections is added to a bottle of the lyophilisate from Example 7. While swirling occasionally, a clear solution is obtained after about 1 min. The solution is virtually isotonic at 305 mosmol/kg.

What is claimed is:

1-53. (canceled)

54. A lyophilisate comprising as an active ingredient flupirtine in base form or as a physiologically tolerated salt and suitable for producing a pharmaceutical composition for parenteral administration.

55. The lyophilisate according to claim 54, comprising at least 100 mg of flupirtine.

56. The lyophilisate according to claim 54, wherein the physiologically tolerated salt is an acid addition salt of flupirtine.

57. The lyophilisate according to claim 56, wherein the acid of the acid addition salt is selected from the group consisting of gluconic acid, formic acid, acetic acid, propionic acid, succinic acid, glycolic acid, lactic acid, malic acid, tartaric acid, citric acid, ascorbic acid, maleic acid, fumaric acid, hydroxymaleic acid, pyruvic acid, phenylacetic acid, benzoic acid, p-aminosalicylic acid, embonic acid, methanesulphonic acid, ethanesulphonic acid, hydroxyethanesulphonic acid, ethylenesulphonic acid, halobenzenesulphonic acid, toluenesulphonic acid, naphthalenesulphonic acid, sulphanilic acid, and hydrochloric acid.

58. The lyophilisate according to claim 56, wherein the acid of the acid addition salt is present in an amount of from 60 mg to 650 mg per 100 mg of flupirtine, preferably in an amount of 200 mg to 400 mg per 100 mg of flupirtine.

59. The lyophilisate according to claim 54, further comprising at least one additive selected from the group consisting of a cake-forming agent, an antioxidant, and a detergent; wherein the cake-forming agent is preferably mannite, saccharose or glycine; wherein the antioxidant is preferably sodium bisulfate or ascorbic acid; and wherein the detergent is preferably poylvinylpyrrolidone.

60. The lyophilisate according to claim 59, wherein the cake-forming agent is present in an amount of from 10 mg to 1000 mg per 100 mg of flupirtine, preferably in an amount of from 30 mg to 300 mg per 100 mg of flupirtine.

61. The lyophilisate according to claim 59, wherein the antioxidant is present in an amount of from 0.5 mg to 10 mg per 100 mg of flupirtine, preferably in an amount of from 2 mg to 5 mg per 100 mg of flupirtine.

62. The lyophilisate according to claim 59, wherein the detergent is present in an amount of from 10 mg to 150 mg per 100 mg of flupirtine, preferably in an amount of from 10 mg to 50 mg per 100 mg of flupirtine.

63. A process for producing a flupirtine-containing pharmaceutical composition for parenteral administration, the process comprising the step of:
dissolving a flupirtine-containing lyophilisate according to claim 54 in at least one solvent, selected from the group consisting of an aqueous medium and an organic solvent, to obtain a liquid pharmaceutical composition ready for use.

64. A process for producing a flupirtine-containing lyophilisate according to claim 54, the process comprising the step of:
preparing a flupirtine solution by dissolving flupirtine base in an aqueous medium;
freeze drying the flupirtine solution.

65. A liquid flupirtine-containing pharmaceutical composition for parenteral administration, prepared by dissolving a flupirtine-containing lyophilisate according to claim 54.

66. The pharmaceutical composition according to claim 65 in the form of a solution for injection or a solution for infusion.
